(19) United States
(12) Patent Application Publication
(10) Pub. No.: US 2020/0405315 A1
(43) Pub. Date: Dec. 31, 2020

(54) LIGATION CLIP WITH RETENTION FEATURES

(71) Applicant: Covidien LP, Mansfield, MA (US)

(72) Inventors: Zhihua Zhang, ShenZhen (CN); Lin Huang, Shanghai (CN)

(21) Appl. No.: 16/978,980

(22) PCT Filed: Mar. 7, 2018
(86) PCT No.: PCT/CN2018/078294

§ 371 Date: Sep. 8, 2020

(51) Int. Cl. A61B 17/122

(52) U.S. Cl. CPC A61B 17/122

(57) ABSTRACT

A surgical ligation clip (10) includes a first jaw (12), a second jaw (14), and a hinge portion (16) that pivotably couples the first jaw (12) to the second jaw (14). The first jaw (12) defines a first clamping surface (22) that supports a longitudinal rib (60). The second jaw (14) defines a second clamping surface (28) that supports first and second rows of protrusions (70a, 70b) that are spaced from each other to define an unobstructed channel (72) to receive the longitudinal rib (60) of the first jaw (12). Each of the protrusions (70a, 70b) may include a sloped distal wall (74) that is configured to facilitate passage of the ligation clip (10) about tissue.

### BACKGROUND

### 1. Technical Description

The present disclosure is directed to ligation clips for sealing body vessels and, more particularly, to ligation clips that include clamping surfaces with tissue retention features for sealing body vessels.

### 2. Background of Related Art

Ligation clips are commonly used during a variety of surgical procedures to ligate tissue, e.g., a body vessel. Typically, ligation clips include first and second jaws that include clamping surfaces. The jaws are pivotably connected to each other at one end and are movable between open and clamped positions. When the ligation clip is applied to a portion of a body vessel, the body vessel is clamped between the clamping surfaces of the first and second jaws. After the ligation clip is applied to the body vessel, any movement of the ligation clip in relation to the body vessel may have a negative impact on the performance of the ligation clip.

A continuing need exists in the art for ligation clips that have clamping surfaces with improved retention characteristics to prevent movement of the ligation clip in relation to a body vessel after the ligation clip is applied to the body vessel.

### SUMMARY

One aspect of the disclosure is directed to a ligation clip that includes a first jaw and a second jaw. The first jaw defines a first clamping surface that supports a central longitudinal rib that extends along at least a portion of the length of the first clamping surface. The second jaw is pivotably supported in relation to the first jaw between an open position and a clamped position and defines a second clamping surface. The second clamping surface of the second jaw includes a first row of protrusions supported on one side of the second clamping surface and a second row of protrusions supported on an opposite side of the second clamping surface. Each of the protrusions in the first row of protrusions is longitudinally aligned with the other protrusions in the first row of protrusions and each of the protrusions in the second row of protrusions is longitudinally aligned with the other protrusions in the second row of protrusions. The first row of protrusions is spaced from the second row of protrusions to define an unobstructed channel that extends longitudinally between the first and second rows of protrusions. The channel is positioned to receive the longitudinal rib. Each of the protrusions in the first and second rows of protrusions includes a sloped distal wall.

In embodiments, each of the protrusions of the first and second rows of protrusions includes a flat upper surface that is spaced from the second clamping surface and defines a plane. The plane of the flat upper surface and the sloped distal wall of each of the protrusions defines an angle Ω, wherein Ω is from about 15 degrees to about 50 degrees.

In some embodiments, Ω is from about 25 degrees to about 40 degrees.

In certain embodiments, Ω is about 35 degrees.

In embodiments, each of the protrusions of the first and second rows of protrusions includes a proximal wall, and the plane of the flat upper surface and the proximal wall of each of the protrusions define an angle β of from about 45 degrees to about 90 degrees.

In some embodiments, β is from about 55 degrees to about 75 degrees.

In certain embodiments, β is about 65 degrees.

In embodiments, the clip is formed of a polymeric material.

In some embodiments, the first and second jaws support bosses configured to support the ligation clip on a clip applier.

In certain embodiments, the first jaw includes a first locking element and the second jaw includes a second locking element, and the second locking element is pivotable into engagement with the first locking element to retain the ligation clip in the clamped position.

In embodiments, the second jaw includes a distal portion that includes spaced teeth that are configured to penetrate tissue.

In some embodiments, the longitudinal rib extends over at least 50 percent of the overall length of the first clamping surface.

In certain embodiments, the longitudinal rib extends over at least 80 percent of the overall length of the first clamping surface.

In embodiments, the protrusions in the first row of protrusions are transversely offset from the protrusions in the second row of protrusions such that the protrusions in the first and second rows of protrusions are alternatingly positioned on opposite sides of the second clamping surface along the length of the second clamping surface.

In some embodiments, the first jaw is pivotably coupled to the second jaw by a living hinge.

In certain embodiments, the longitudinal rib has a rectangular configuration.

In embodiments, the first jaw includes a third row of protrusions positioned on the first clamping surface on one side of the central longitudinal rib and a fourth row of protrusions positioned on the first clamping surface on the other side of the central longitudinal rib.

In some embodiments, each of the protrusions in the third row of protrusions is positioned to be received between two adjacent protrusions in the first row of protrusions when the ligation clip is in the clamped position.

In certain embodiments, each of the protrusions in the fourth row of protrusions is positioned to be received between two adjacent protrusions in the second row of protrusions when the ligation clip is in the clamped position.

In embodiments, the protrusions in the third and the fourth rows of protrusions have sloped proximal and distal walls.

### BRIEF DESCRIPTION OF THE DRAWINGS

Various exemplary embodiments of the presently disclosed ligation clip are described herein below with reference to the drawings, wherein:

FIG. 1 is a side perspective view of an exemplary embodiment of the presently disclosed ligation clip in an open position;

FIG. 2 is an enlarged view of the indicated area of detail shown in FIG. 1;

FIG. 2A is an enlarged view of the indicated area of detail shown in FIG. 2;

FIG. 3 is a side perspective view of the ligation clip shown in FIG. 1 in the clamped position;

FIG. 3A is a cross-sectional view taken along section line 3A-3A of FIG. 3;

FIG. 4 is an enlarged view of the indicated area of detail shown in FIG. 3;

FIG. 5 is a side view of the ligation clip shown in FIG. 1 in the open position positioned about tissue;

FIG. 5A is a perspective view of the ligation clip shown in FIG. 1 as the clip is moved from the open position towards the clamped position;

FIG. 6 is a side view of the ligation clip shown in FIG. 5 placed about tissue in the clamped position; and

FIG. 7 is an enlarged view of the indicated area of detail shown in FIG. 6;

FIG. 8 is a side perspective view of a proximal portion of another exemplary embodiment of the presently disclosed ligation clip in an open position;

FIG. 9 is a side view of the ligation clip shown in FIG. 8 in the clamped position; and

FIG. 10 is a cross-sectional view taken along section line 10-10 of FIG. 9.

### DETAILED DESCRIPTION OF EMBODIMENTS

The presently disclosed ligation clip will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel. Further, the term “about” is used generally to mean plus or minus 10 percent of the disclosed parameter.

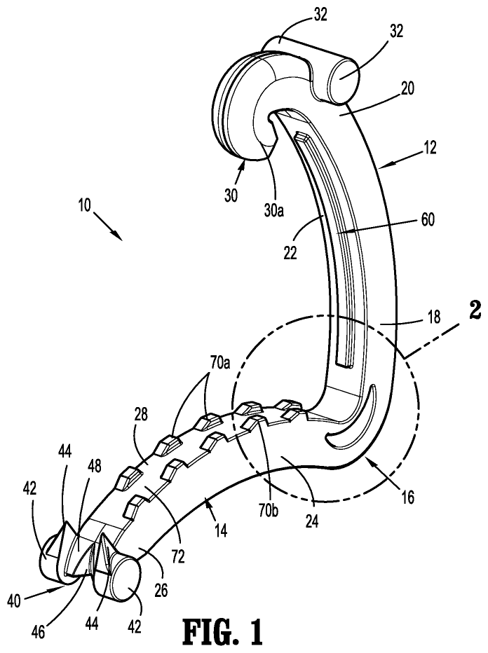

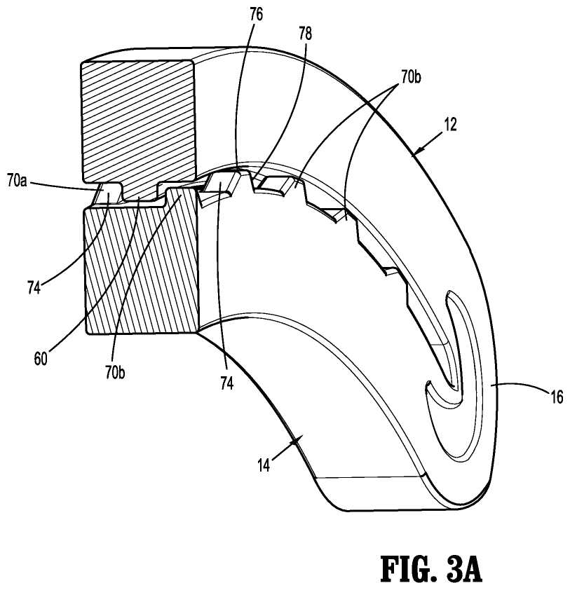

Referring to FIGS. 1-3A, an exemplary embodiment of the presently disclosed ligation clip is shown generally as 10 and includes a first jaw 12, a second jaw 14, and a hinge portion 16 coupling the first jaw 12 to the second jaw 14. The first jaw 12 is pivotable in relation to the second jaw 14 about the hinge portion 16 to move the ligation clip 10 between an open position (FIG. 1) and a clamped position (FIG. 3). In embodiments, the first and second jaws 12, 14 are curved along the longitudinal axis of the ligation clip 10 although other jaw configurations are envisioned. The hinge portion 16 may be integrally formed with the first and second jaws 12, 14, e.g., a living hinge, and may define a crescent shaped through bore 16a to facilitate movement of the first jaw 12 in relation to the second jaw 14 between the open and clamped positions. The through bore 16a also allows for substantially complete closure of proximal portions of the first and second jaws 12, 14.

The first jaw 12 includes a proximal portion 18, a distal portion 20, and a clamping surface 22. The second jaw 14 includes a proximal portion 24, a distal portion 26, and a clamping surface 28. The proximal portions 18, 24 of the first and second jaws 12, 14, respectively, are coupled to or integrally formed with the hinge portion 16.

The distal portion 20 of first jaw 12 includes a first locking element 30 and spaced bosses 32. The first locking element 30 includes a hooked portion 30a that extends downwardly and proximally from the tissue clamping surface 22 to define a portion of a latching mechanism that is configured to retain the ligation clip 10 in a clamped position as described in further detail below. The bosses 32, which may form part of a single cylindrical member supported on the first jaw 12, are positioned and configured to engage the jaws of an applicator (not shown) to facilitate application of the ligation clip 10 to tissue.

The distal portion 26 of the second jaw 14 includes a second locking element 40, spaced bosses 42, and spaced teeth 44. The spaced teeth 44 are configured to penetrate tissue. In embodiments, the teeth 44 have an apex or pointed end 44a that grips and stretches tissue as the ligation clip 10 is moved from the open position (FIG. 1) towards the clamped position (FIG. 3) to improve the ligating characteristics of the ligation clip 10. The second locking element 40 defines a recess 46 (FIG. 1) and a cam surface 48. The cam surface 48 and the recess 46 are configured to receive and guide the first locking element 30 into locking engagement with the second locking element 40 when the ligation clip 10 is moved to the clamped position (FIG. 3) to secure the ligation clip 10 in the clamped position. In particular, the cam surface 48 is configured to deflect the first locking element 30 outwardly in a distal direction as the first locking element 30 passes over the cam surface 48 such that when the first locking element 30 moves past the cam surface 48, the first locking element 30 resiliently moves into engagement with the second locking element 40. The bosses 42 of the second jaw 14 are similar to the bosses 32 of the first jaw 12 and are configured to engage jaws of an applicator (not shown) to facilitate application of the ligation clip 10 to tissue.

The first clamping surface 22 is substantially flat and supports a longitudinal rib 60. In embodiments, the longitudinal rib 60 has a substantially rectangular configuration and includes a surface 62 in opposition to the clamping surface 28 of the second jaw 14. In embodiments, the surface 62 is flat and is spaced from and substantially parallel to the clamping surface 22. In some embodiments, the surface 62 is continuous and extends substantially the entire length, e.g., greater than 80 percent of the overall length, of the tissue clamping surface 22. It is envisioned that the longitudinal rib 60 may include one or more rib sections that are longitudinally spaced from each other and may extend over a length less than 80 percent of the overall length of the tissue clamping surface 22, e.g., 50-80 percent of the overall length of the tissue clamping surface 22. It is also envisioned that the surface 62 of the longitudinal rib 60 need not be flat but may be curved, ribbed, knurled, or otherwise configured to enhance tissue retention.

The second clamping surface 28 is substantially flat and supports a first row of protrusions 70a and a second row of protrusions 70b. Each of the protrusions 70a of the first row of protrusions 70a is spaced from but longitudinally aligned with each of the other protrusions 70a in the first row along a first side of the second clamping surface 28. Similarly, each of the protrusions 70b of the second row of protrusions 70b is longitudinally aligned with each of the other protrusions 70b in the second row along a second side of the second clamping surface 28. The protrusions 70a, 70b each have a width that is less than half the width of the clamping surface 28 to define an unobstructed central channel 72 between the first row of protrusions 70a and the second row of protrusions 70b on the second clamping surface 28. The central channel 72 is dimensioned to receive the longitudinal rib 60 of the first jaw 12 when the ligation clip 10 is in the clamped position. In embodiments, the protrusions 70a on the first side of the clamping surface 28 are transversely offset from the protrusions 70b positioned on the other side of the clamping surface 28 such that the protrusions 70a and 70b are alternatingly positioned on opposite sides of the clamping surface 28 along the length of the clamping surface 28.

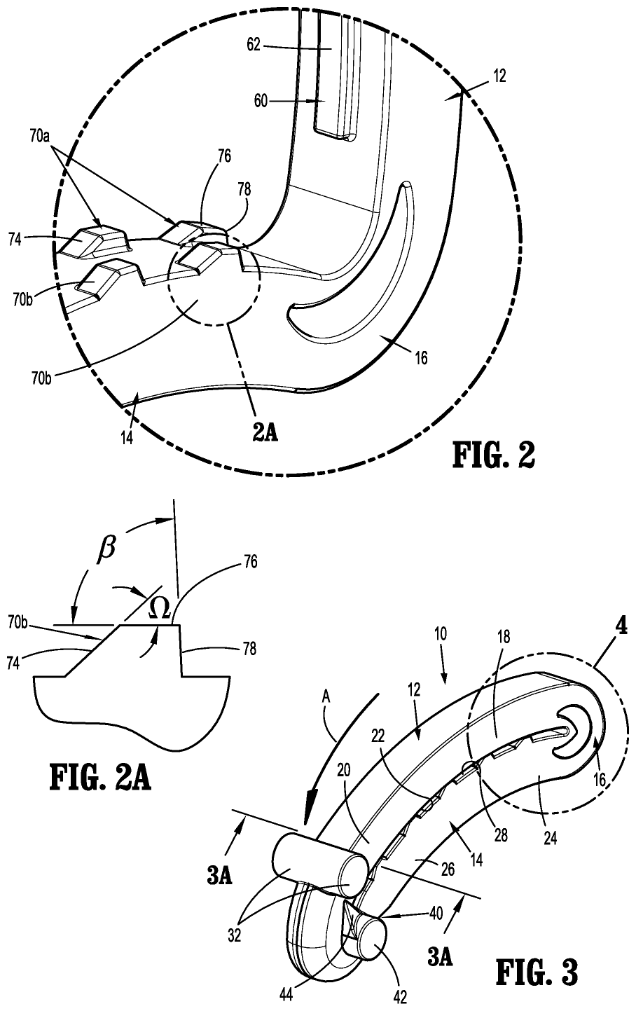

Referring to FIG. 2A, each of the protrusions 70a and 70b (only a protrusion 70b is shown) includes a tapered or sloped distal wall 74, a flat surface 76, and a proximal wall 78 that is more vertical than the distal wall 74 as viewed in FIG. 2A. The sloped distal wall 74 and a plane defined by the surface 76 define an angle Ω and the proximal wall 78 and the plane defined by the surface 76 define an angle β. In embodiments, Ω is from about 15 degrees to about 50 degrees. In some embodiments, Ω is from about 25 degrees to about 40 degrees. In certain embodiments, Ω is about 35 degrees. In embodiments, β is from about 45 degrees to about 90 degrees. In some embodiments, β is from about 55 degrees to about 75 degrees. In certain embodiments, β is about 65 degrees.

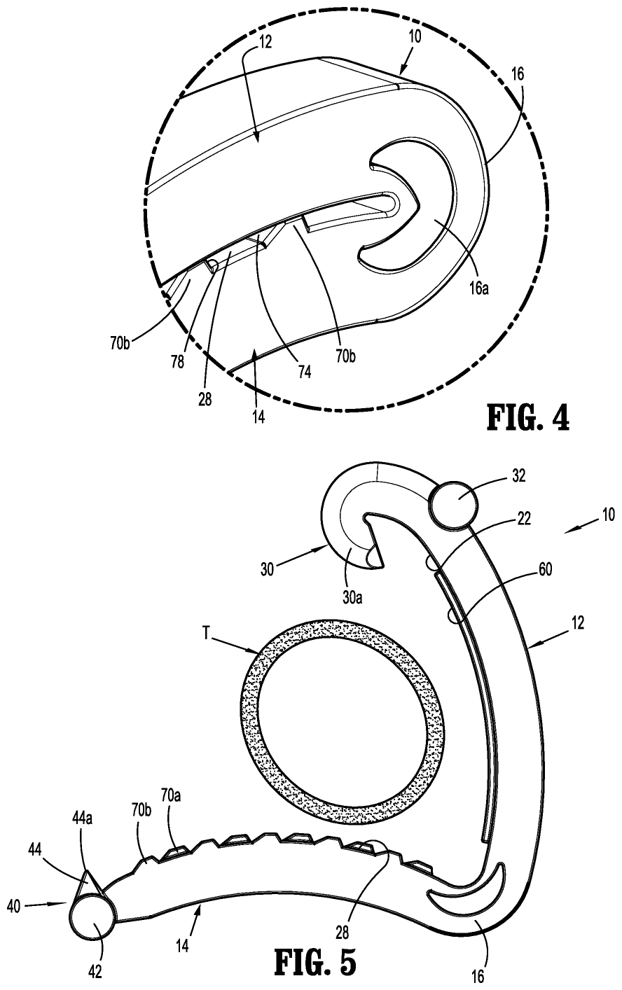

Referring to FIGS. 3-4, when the first jaw 12 and the second jaw 14 are moved from the open position (FIG. 1) to the clamped position in the direction indicated by arrow A in FIG. 3, the first jaw 12 pivots in relation to the second jaw 14 about the hinge 16 to move the hooked portion 30a of the first locking element 30 into engagement with the second locking element 40 to secure the ligation clip 10 in the clamped position. In the clamped position, to the longitudinal rib 60 of the clamping surface 22 of the first jaw 12 is received in the central channel 72 defined between the first and second rows of protrusions 70a and 70b. As shown in FIG. 3A, in the clamped position, the protrusions 70a and 70b are transversely offset from each other.

In embodiments, the ligation clip 10 may be made at least in part of a resilient bioabsorbable polymeric material. Examples of suitable bioabsorbable polymeric materials include acetal polyoxymethylene (POM), polyethylene terephthalate (PET), polybutylene terephthalate (PBT), polyoxymethylene, polyetheretherketone (PEEK), polypropylene, and polyethylene or other thermoplastic materials having similar properties that can be injection-molded. The ligation clip 10 may also be made at least in part of a polymeric material or materials in combination with radiolucent metal alloys. Alternately, other materials may be used to form the ligation clip 10 including biocompatible metals, plastics and composites.

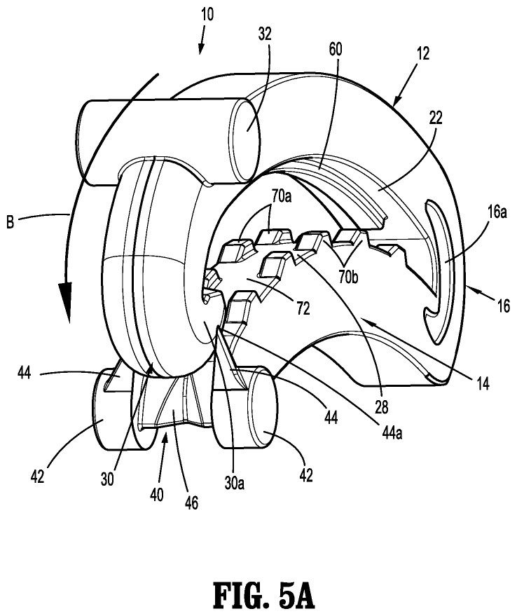

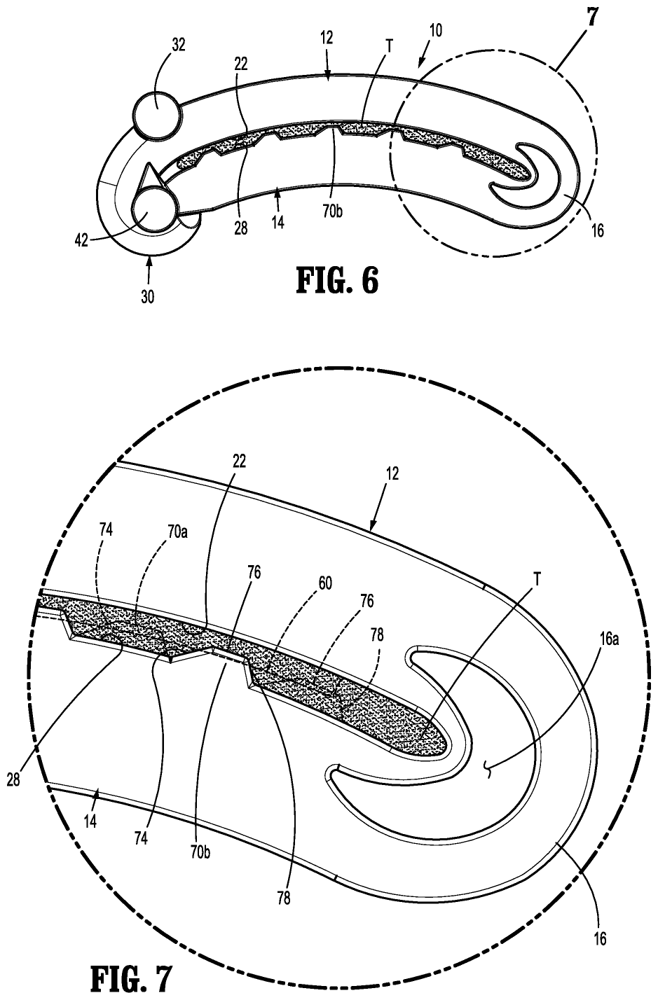

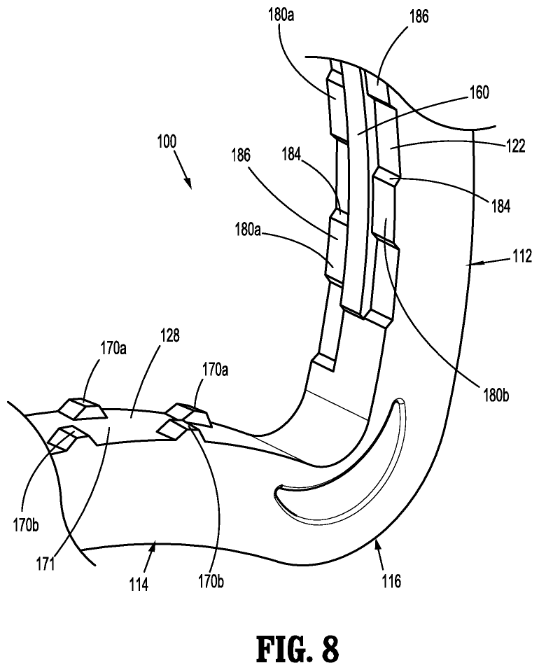

Referring to FIGS. 5-7, in use, the ligation clip 10 is positioned about tissue “T”, e.g., a body vessel, such that the tissue “T” is positioned between the tissue clamping surface 22 of the first jaw 12 and the tissue clamping surface 28 of the second jaw 14. As described above, when the ligation clip 10 is moved from the open position (FIG. 1) to the closed position (FIG. 6) in the direction indicated by arrow “B” in FIG. 5A, the first jaw 12 pivots in relation to the second jaw 14 about the hinge 16 to move the hooked portion 30a of the first locking element 30 into engagement with the second locking element 40 to secure the ligation clip 10 in the clamped position. As shown in FIG. 7, in the clamped position, the tissue “T” is compressed between the first and second clamping surfaces 22, 28 of the first and second jaws 12, 14, respectively. More particularly, when the ligation clip 10 is moved to the clamped position, the tissue “T” is compressed between the upper surface 76 of the protrusions 70a, 70b and the tissue clamping surface 22 of the first jaw, and to a lesser extent between the distal and proximal walls 74, 78, of the protrusions 70a, 70b and the tissue clamping surface 22 of the first jaw 12 at multiple spaced locations along the ligation clip 10. In addition, the tissue “T” is also compressed between the surface 62 of the longitudinal rib 60 and the clamping surface 28 of the second jaw 14, and between the opposed clamping surfaces 22, 28 of the first and second jaws 12, 14, respectively.

The above described ligation clip 10 including the first row of protrusions 70a, the second row of protrusions 70b, and the longitudinal rib 60 enhances the retention forces of the ligation clip 10 on the tissue “T” and minimizes the likelihood that the ligation clip 10 will slide in a direction parallel to the clamping surfaces 22, 28 of the first and second jaws 12, 14, respectively, about the tissue after the ligation clip 10 has been clamped about the tissue “T”. The wedge shape of the protrusions 70a, 70b helps to locate or position the tissue “T” within the ligation clip 10 during placement of the ligation clip 10 about the tissue “T”. More specifically, the sloped distal walls 74 of the protrusions 70a, 70b allow the ligation clip 10 to more easily pass over the tissue “T” during placement of the ligation clip 10 about the tissue “T”.

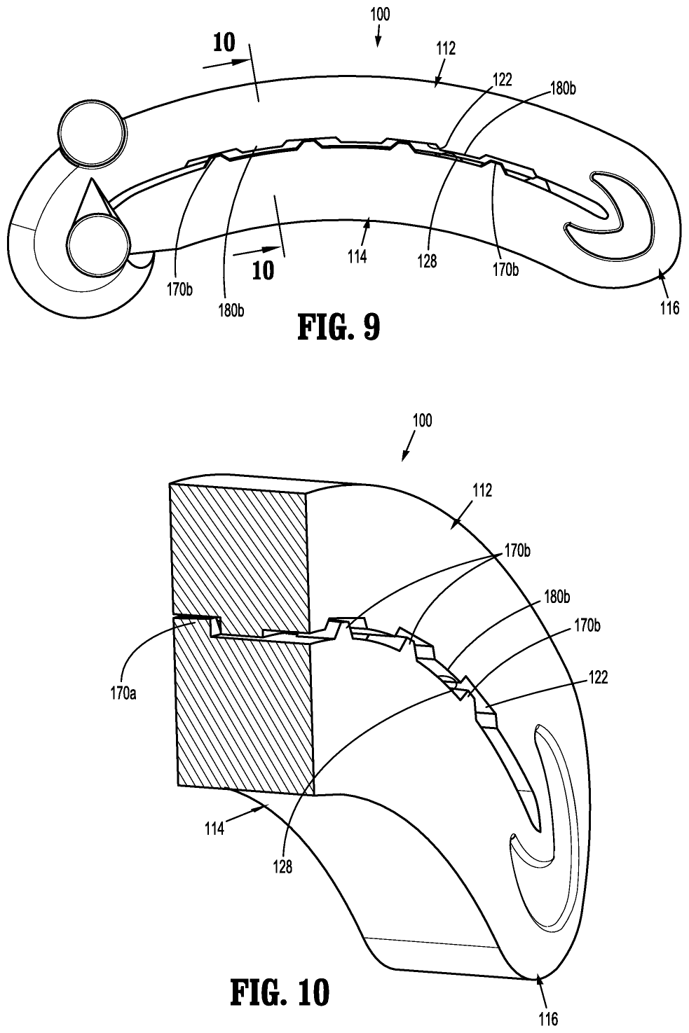

FIGS. 9-11 illustrate another exemplary embodiment of the presently disclosed ligation clip shown generally as ligation clip 100. Ligation clip 100 is similar to ligation clip 10 (FIG. 1) in most respects and includes a first jaw 112, a second jaw 114 and a hinge portion 116. The first jaw 112 includes a clamping surface 122 that supports a longitudinal rib 160. The second jaw 114 includes a clamping surface 128 that supports a first row of projections 170a positioned along a first edge of the clamping surface 128 and a second row of projections 170b positioned along an opposite side of the clamping surface 128. The first and second rows of protrusions 170a, 170b define a central channel 171 that is dimensioned to receive the longitudinal rib 160 when the ligation clip 100 is in a clamped position.

Ligation clip 100 differs from ligation clip 10 in that the first jaw 112 also includes a first row of protrusions 180a that are positioned on one side of the longitudinal rib 160 and a second row of protrusions 180b that are positioned on the other side of the longitudinal rib 160. Each of the protrusions 180a, 180b includes tapered or sloped proximal and distal walls 184 and a flat tissue engaging surface 186 that is in opposition to the clamping surface 128 of the second jaw when the ligation clip 100 is in the clamped position. In embodiments, each of the protrusions 180a in the first row of protrusions 180a on the first jaw 112 is positioned to be received between two protrusions 170a in the first row of protrusions 170a on the second jaw 114 when the ligation clip 100 is in the clamped position. Similarly, each of the protrusions 180b in the second row of protrusions 180b on the first jaw 112 is positioned to be received between two protrusions 170b in the second row of protrusions 170b on the second jaw 1114 when the ligation clip 100 is in the clamped position.

The ligation clip 100 functions in a substantially similar manner to the ligation clip 10 described above except that the ligation clip 100 provides additional tissue retention between the tissue engaging surfaces 186 of the protrusions 180a, 180b of the first jaw 112 and the tissue clamping surface 128 and protrusions 170a, 170b of the second jaw 114.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A ligation clip comprising:
a first jaw defining a first clamping surface, the first clamping surface supporting a central longitudinal rib that extends along at least a portion of the length of the first clamping surface; and
a second jaw pivotably supported in relation to the first jaw and defining a second clamping surface, the second clamping surface of the second jaw having a first row of protrusions supported on one side of the second clamping surface and a second row of protrusions supported on an opposite side of the second clamping surface, the protrusions in the first row of protrusions being longitudinally aligned and the protrusions in the second row of protrusions being longitudinally aligned, the first row of protrusions being spaced from the second row of protrusions to define an unobstructed channel that extends longitudinally between the first and second rows of protrusions, the channel being positioned to receive the longitudinal rib, wherein each of the protrusions in the first and second rows of protrusions includes a sloped distal wall.

2. The ligation clip of claim 1, wherein each of the protrusions of the first and second rows of protrusions includes a flat upper surface that is spaced from the second clamping surface and defines a plane, the plane of the flat upper surface and the sloped distal wall of each of the protrusions defining an angle Ω, wherein Ω is from about 15 degrees to about 50 degrees.

3. The ligation clip of claim 2, wherein Ω is from about 25 degrees to about 40 degrees.

4. The ligation clip of claim 2, wherein Ω is about 35 degrees.

5. The ligation clip of claim 2, wherein each of the protrusions of the first and second rows of protrusions includes a proximal wall, the plane of the flat upper surface and the proximal wall of each of the protrusions defining an angle β, wherein β is from about 45 degrees to about 90 degrees.

6. The ligation clip of claim 5, wherein β is from about 55 degrees to about 75 degrees.

7. The ligation clip of claim 6, wherein β is about 65 degrees.

8. The ligation clip of claim 1, wherein the ligation clip is formed of a polymeric material.

9. The ligation clip of claim 1, wherein the first and second jaws support bosses, the bosses being configured to engage a clip applier.

10. The ligation clip of claim 1, wherein the first jaw includes a first locking element and the second jaw includes a second locking element, the second locking element being pivotable into engagement with the first locking element to retain the ligation clip in the clamped position.

11. The ligation clip of claim 1, wherein the second jaw includes a distal portion, the distal portion including spaced teeth that are configured to penetrate tissue.

12. The ligation clip of claim 1, wherein the longitudinal rib extends over at least 50 percent of the overall length of the first clamping surface.

13. The ligation clip of claim 12, wherein the longitudinal rib extends over at least 80 percent of the overall length of the first clamping surface.

14. The ligation clip of claim 1, wherein the protrusions in the first row of protrusions are transversely offset from the protrusions in the second row of protrusions such that the protrusions are alternatingly positioned on opposite sides of the second clamping surface along the length of the second clamping surface.

15. The ligation clip of claim 1, wherein the first jaw is pivotably coupled to the second jaw by a living hinge.

16. The ligation clip of claim 1, wherein the longitudinal rib has a rectangular configuration.

17. The ligation clip of claim 1, wherein the first jaw includes a third row of protrusions positioned on the first clamping surface on one side of the central longitudinal rib and a fourth row of protrusions positioned on the first clamping surface on the other side of the central longitudinal rib.

18. The ligation clip of claim 17, wherein each of the protrusions in the third row of protrusions is positioned to be received between two adjacent protrusions in the first row of protrusions when the ligation clip is in the clamped position.

19. The ligation clip of claim 18, wherein each of the protrusions in the fourth row of protrusions is positioned to be received between two adjacent protrusions in the second row of protrusions when the ligation clip is in the clamped position.

20. The ligation clip of claim 18, wherein the protrusions in the third and the fourth rows of protrusions have sloped proximal and distal walls.
